(19) United States
(12) Patent Application Publication
(10) Pub. No.: US 2023/0092955 A1
(43) Pub. Date: Mar. 23, 2023

(54) Refillable Implantable Device for Delivering a Drug Compound

(71) Applicant: Celanese EVA Performance Polymers LLC, Irving, TX (US)

(72) Inventors: Vijay Gyanani, Powell, OH (US); Jeffrey Haley, Norwood, OH (US); Keith Henry, Middleton, DE (US); Kevin Welsh, Worthville, KY (US)

(21) Appl. No.: 17/948,271

(22) Filed: Sep. 20, 2022

Related U.S. Application Data
(60) Provisional application No. 63/305,772, filed on Feb. 2, 2022.
(60) Provisional application No. 63/246,844, filed on Sep. 22, 2021.

(51) Int. Cl. A61M 5/142; A61P 15/18; A61F 6/14; A61K 31/566; A61K 31/57

(52) U.S. Cl. CPC A61M 5/14276; A61P 15/18; A61F 6/14; A61K 31/566; A61K 31/57; A61M 2209/045

(57) ABSTRACT

An implantable and refillable device for delivering a contraceptive agent is provided. The device comprises a reservoir within which the contraceptive agent is capable of being retained, wherein the reservoir defines a first surface and a second surface opposing the first surface. A release structure comprising a hydrophobic polymer surrounds at least a portion of the reservoir. The release structure is in communication with the reservoir such that the contraceptive agent can pass from the reservoir through the release structure. A septum is positioned adjacent to the first surface of the reservoir and a backing layer is positioned adjacent to the second surface of the reservoir.

### RELATED APPLICATIONS

The present application is based upon and claims priority to U.S. Provisional Patent Application Ser. No. 63/246,844, having a filing date of Sep. 22, 2021 and U.S. Provisional Patent Application Ser. No. 63/305,772, having a filing date of Feb. 2, 2022, which are incorporated herein by reference.

### BACKGROUND

Implantable medical devices have been used for some time to provide long-term and reversible contraception. Unfortunately, many implantable devices can carry risks and complications including dosing errors, particularly for contraceptive agents that have been traditionally difficult to controllably deliver over a sustained period of time. Attempts have been made to make implantable delivery devices refillable for extended controlled delivery of contraceptive agents. This has been met with difficulties as well, including infection arising from the port system used for refilling the device reservoir and an inconsistent drug delivery rate due to the need for multiple device refilling cycles. As such, a need continues to exist for an implantable and refillable delivery device that is capable of delivering a contraceptive agent over a sustained period of time.

### SUMMARY OF THE INVENTION

In accordance with one embodiment of the present disclosure, an implantable and refillable device is disclosed. The device a reservoir within which a contraceptive agent is capable of being retained, wherein the reservoir defines a first surface and a second surface opposing the first surface. A release structure comprising a hydrophobic polymer surrounds at least a portion of the reservoir. The release structure is in communication with the reservoir such that the contraceptive agent can pass from the reservoir through the release structure. Further, a septum is positioned adjacent to the first surface of the reservoir and a backing layer is positioned adjacent to the second surface of the reservoir.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of an implantable medical device of the present disclosure;

FIG. 2 is a cross-sectional view of the implantable medical device of FIG. 1;

FIG. 3 is a perspective view of another embodiment of an implantable medical device of the present disclosure;

FIG. 4 is a cross-sectional view of the implantable medical device of FIG. 3; and

FIG. 5 is a cross-sectional view of a multi-layered septum as may be included in a device as disclosed herein.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to an implantable device that is capable of delivering a contraceptive agent (e.g., contraceptive) to a patient (e.g., human, pet, farm animal, etc.) over a sustained period of time to help prohibit and/or treat a condition, disease, and/or cosmetic state of the patient. The device includes a reservoir within which the contraceptive agent is capable of being retained and a release structure that is in communication with the reservoir such that the contraceptive agent can pass from the reservoir through the release structure. The device also includes a septum that can be used for refilling the reservoir of the device and a backing layer opposite the septum that can inhibit needle penetration through the device during refilling.

The implantable device may have a variety of different geometric shapes, such as circular or ovoid disc, cylindrical (rod), ring, doughnut, helical, elliptical, triangular, ovular, etc. In one embodiment, for example, the device may have a generally circular cross-sectional shape so that the overall structure is in the form of a disc or rod. In such embodiments, the device will typically have a diameter of from about 0.5 to about 3 centimeters, such as from about 0.75 to about 2 centimeters, in some embodiments from about 0.8 to about 1.5 centimeters. The height of the device may vary, but is typically in the range of from about 0.25 to about 1.5 centimeters. In some embodiments, a reservoir of a device can define a volume of from about 0.05 to about 5 milliliters, for instance from about 1 to about 4 milliliters. Regardless of the particular size or shape, the device includes a release structure adjacent to the reservoir and through which one or more contraceptive agents can pass.

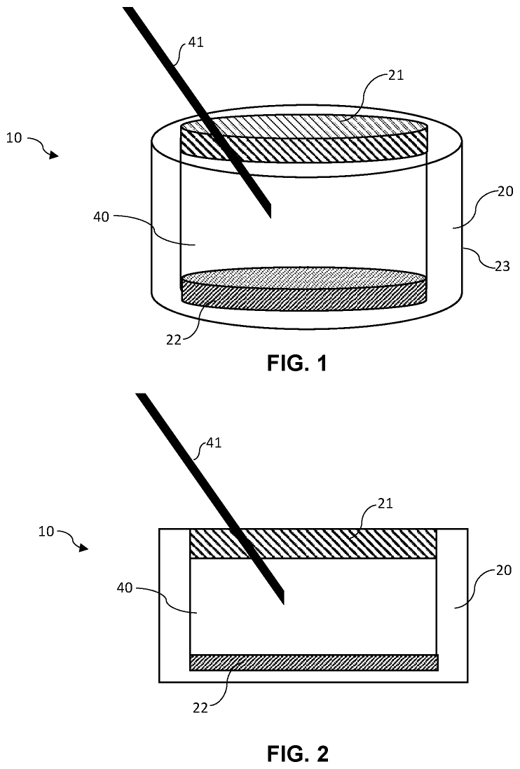

Referring to FIG. 1 and FIG. 2, for example, one embodiment of an implantable device 10 is shown that contains a reservoir 40 having a generally circular cross-sectional shape and that is generally disc-shaped in nature. A release structure 20 likewise surrounds at least a portion of the reservoir 40, and in some cases, the entire reservoir 40. The device also includes a septum 21 that is positioned adjacent to a first surface (e.g., upper surface) of the reservoir 40 and a backing layer 22 that is positioned adjacent to a second opposing surface (e.g., lower surface) of the reservoir 40. As shown, the release structure 20 may also surround at least a portion of the septum 21 and/or at least a portion of the backing layer 22. During use of the device 10, the contraceptive agent is capable of being released from the reservoir 40 by passage through the release structure 20 so that it exits from an external surface 23 of the device. When it is desired to refill the device, a needle 41 can penetrate the septum and be inhibited by the backing layer 22 from over-penetration and passage completely through and out of the reservoir. The contraceptive agent can thus be injected into the reservoir for refilling.

Of course, in other embodiments, the release structure may contain multiple layers. In the device of FIG. 1 and FIG. 2, for example, one or more additional layers (not shown) may be disposed over the layer 20 to help further control release of the contraceptive agent.

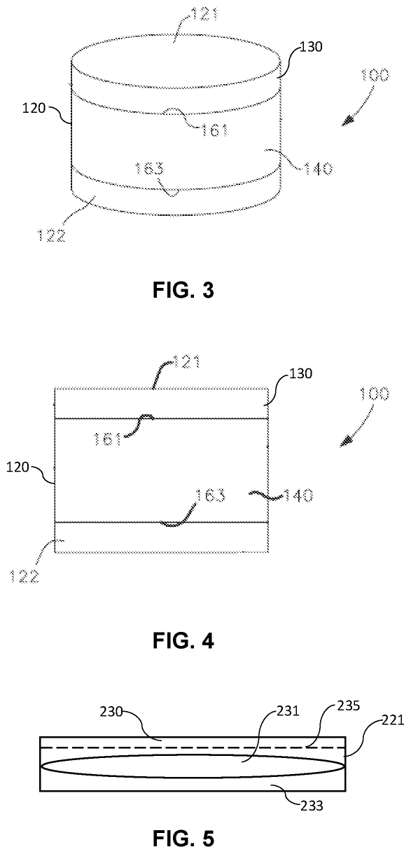

The device may be configured so that the backing layer is held within the release structure or alternatively forms an outer surface of the device. Referring to FIG. 3 and FIG. 4, for example, one embodiment of an implantable device 100 is shown that contains a reservoir 140 having a generally circular cross-sectional shape and a height so that the resulting device is generally disc-shaped in nature. The reservoir 140 is surrounded by a release structure 120 that has an upper edge 161 to which is adhered a septum 121 and a lower edge 163 to which is adhered a backing layer 122. As shown, the septum 121 and the backing layer 122 can have a generally circular cross-sectional shape and form an upper and lower surface of the device 100. If desired, a portion of the release structure 120 may also extend beneath a lower surface of the septum 121 and along an upper surface of the backing layer 122, which can improve the seal between the materials and prevent any uncontrolled leakage of the contents of the reservoir 140. During use of the device 100, the contraceptive agent is capable of being released from the reservoir 140 and through the release structure 120 so that it exits from the device. Of course, if desired, one or more additional layers (not shown) may also be disposed over the release structure 120 to help further control release of the contraceptive agent.

Through selective control over the particular nature of the release structure, septum, and/or backing layer, as well as other aspects of the device, it is believed that the resulting device can be effective for sustained release of one or more contraceptive agents over a prolonged period of time. For example, upon a single filling of the reservoir, the implantable device can release the contraceptive agent for a time period of about 5 days or more, in some embodiments about 10 days or more, in some embodiments from about 20 days to about 60 days, and in some embodiments, from about 25 days to about 50 days (e.g., about 30 days) or even longer in some embodiments. For instance, a device need only be refilled on a month-long time scale, e.g., once a month, bimonthly, once in three months, once in four months, once in six months, or more. In some embodiments, a device can be designed to be refilled once a year, or even longer.

Through refilling of the reservoir, the implantable device can release the contraceptive agent from several weeks to several months in some embodiments. For instance, the effective drug delivery time period over the entire course of use (i.e., including refilling of the device) can be in the range of about three months to multiple years, e.g., about six months to about six years; about six months to about five years; or about one year to about four years, such as about 18 months in some embodiments. Further, the contraceptive agent can also be released in a controlled manner (e.g., zero order or near zero order) over the course of the release time period. Moreover, through selective control over the particular nature of the release structure, septum, and/or backing materials used in forming the device, the device can be refilled multiple times without loss of desirable release characteristics.

Various embodiments of the present disclosure will now be described in more detail.

### I. Release Structure

As indicated above, the device includes a release structure through which the contraceptive agent can be delivered from a reservoir to a surrounding area, e.g., via passive diffusion through the release structure. The release structure generally contains a polymeric matrix that is formed from at least one polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the release structure polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms.

In certain embodiments, the release structure may contain a semi-crystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may likewise be within a range of from about 40 wt. % to about 90 wt. %, in some embodiments about 40 wt. % to about 80 wt. %, and in some embodiments, from about 50 wt. % to about 75 wt. %.

In one particular embodiment, for example, the release structure may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the release structure polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and release structure have a melting temperature and/or melt flow index within the range noted above. For example, the release structure may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the release structure.

In certain cases, ethylene vinyl acetate copolymer(s) constitute the entire hydrophobic polymer content of the release structure. In other cases, however, it may be desired to include other polymers, such as other hydrophobic polymers. When employed, it is generally desired that such other hydrophobic polymers constitute from about 0.001 wt. % to about 30 wt. %, in some embodiments from about 0.01 wt. % to about 20 wt. %, and in some embodiments, from about 0.1 wt. % to about 10 wt. % of the hydrophobic polymer content of the release structure polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the hydrophobic polymer content of the release structure.

When employing multiple release structure layers, it is typically desired that each layer contains a polymer matrix that includes a hydrophobic polymer. For example, a first release structure layer may contain a first polymer matrix and a second release structure layer may contain a second polymer matrix. In such embodiments, the first and second polymer matrices each contain a hydrophobic polymer, which may be the same or different. In one embodiment, for instance, all of the release structure layer(s) employ the same hydrophobic polymer (e.g., α-olefin copolymer). In yet other embodiments, release structure layers may employ different hydrophobic polymers, for instance a first release structure layer may employ a hydrophobic polymer (e.g., α-olefin copolymer) that has a lower melt flow index than a polymer employed in a second release structure layer. Among other things, this can further help control the release of the contraceptive agent from the device.

The release structure may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a release structure layer.

To help control the release rate from the implantable medical device, a hydrophilic compound may also be incorporated into the release structure that is soluble and/or swellable in water. The weight ratio of the hydrophobic polymers, e.g., ethylene vinyl acetate copolymer(s), to the hydrophilic compounds within the release structure may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the polymer matrix, while hydrophobic polymers typically constitute from about 40 wt. % to about 99 wt. %, in some embodiments from about 50 wt. % to about 98 wt. %, and in some embodiments, from about 60 wt. % to about 95 wt. % of a release structure layer.

Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the release structure can include a plurality of water-soluble particles distributed within the polymer matrix. The particle size of the water-soluble particles can be controlled to help achieve the desired delivery rate. For instance, the median diameter (D50) of the particles can be about 100 micrometers or less, in some embodiments about 80 micrometers or less, in some embodiments about 60 micrometers or less, and in some embodiments, from about 1 to about 40 micrometers, such as may be determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles can also be selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a release structure layer.

One or more nonionic, anionic, and/or amphoteric surfactants may also be employed to help create a uniform dispersion of materials in the release structure. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of a release structure layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

Regardless of the particular components employed, the release structure may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the polymeric matrix (e.g., hydrophobic polymer, hydrophilic compound(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, polar comonomer units (e.g., vinyl acetate) can serve as an “internal” plasticizer by inhibiting crystallization of the olefin chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20° C. to about 200° C., in some embodiments, from about 30° C. to about 150° C., in some embodiments from about 40° C. to about 100° C., and in some embodiments, in some embodiments from about 100° C. to about 120° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or contraceptive agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the die (e.g., extruder die) through which the melted polymer flows.

Once melt blended together, the resulting polymer composition may be in the form of pellets, sheets, fibers, filaments, etc., which may be shaped into the desired shape (e.g., a cylinder, a sac, etc.) using a variety of known techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the reservoir. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al.

### II. Septum

As indicated above, an implantable device can include a septum for refilling the device following implantation. The septum may include a “self-sealing” material that has sufficient properties to allow it to close when a hole is formed by a needle therein. For example, the material used to form the septum may be resilient and can exhibit some elasticity, but is generally firm enough to enable detection of the device beneath the skin by palpation. In other words, the material may be flexible and possess a sufficient degree of hardness without excessive stiffness so as to be self-sealing at thickness of about 1 millimeter or greater. In one embodiment, the septum can include a medical grade silicone polymer. In alternative embodiments, the septum can include other medical grade elastomers or rubbers as are known in the art as well as combinations of materials. Suitable self-sealing materials can include, without limitation, nitrile rubbers, styrene block copolymers, thermoplastic or thermoset polyurethanes, or combinations thereof. In some embodiments, a self-sealing material can exhibit a relatively low hardness, such as a Shore A hardness of about 40 or less, in some embodiments from about 10 to about 30, and in some embodiments, from about 10 to about 20 as determined according to ASTM D2240-15 (2021). Silicone elastomers, such as a dimethyl or dimethyl-diphenyl silicone elastomer, may be used in forming a single-layer or multi-layer septum.

In certain embodiments as illustrated in FIG. 3 and FIG. 4, the septum 121 can be secured to a surrounding clamp or fastener in the form of an encircling retainer ring 130 that can exert radial pressure on the septum 121 and enhance the self-sealing characteristics of the material. The effectiveness of the septum 121 can be improved by the radial compressive force applied to the septum 121 by the retainer ring 130. In general, the greater the radial compressive force applied to septum 121, the higher the self-sealing effectiveness of the septum 121. A retainer ring can be formed of any biocompatible and implantable material capable of exerting desired compressive strength, such as stainless steel, titanium, or the like. In such an embodiment, the septum 121 can include features to allow securement. Such features may include tabs, ridges, recesses, or any other suitable securement features known in the art. In some embodiments, securement between a septum 121 and a retainer ring 130 can be through utilization of a biocompatible adhesive, ultrasonic welding, or combinations of materials and securement methodologies. In some embodiments, the release structure polymer layer 120 can be secured to the septum 121 in conjunction with the clamp or retainer ring 130. By way of example, a clamp (e.g., a retainer ring 130), can be used to compress and secure the release structure polymer 120 against the septum 121 as well as to exert a radial pressure on the septum 121.

The septum 121 may also include a single layer of material or multiple layers of materials placed adjacent to each other. FIG. 5 illustrates a cross-sectional view of one embodiment of a septum 221 that includes multiple layers of material. For instance, a septum 221 can include an interior layer 231 such as an intermediate silicone gel layer encased between two self-sealing elastomer layers 230, 233. A silicone gel layer 231 can aid the septum 221 to seal a hole formed by a penetrating needle and prevent uncontrolled release of fluid from the reservoir.

A septum 121, 221 can optionally include additional layers, such as a mesh layer 235 as in FIG. 5 that may be integrated, or embedded, within a polymeric layer, e.g., elastomer layers 230. A mesh layer 235 can enhance the self-sealing characteristics of a polymeric layer, and can add strength to the septum, allowing the device to maintain desired shape upon manipulation, insertion of needles, and contact with other elements of the surrounding environment (e.g., other parts of the interior of the patient's body, or forces exerted from outside the patient's body). In some embodiments, a mesh layer 235 may act in combination with a gel layer 231 to enhance the self-sealing characteristics of a septum 221. For example, after a void is created in the septum 221 by a needle, the gel layer 231 can prevent fluid from having a direct path across the septum 221 and the mesh layer 235 can enhance this property of the gel layer 231 by physically constraining the gel from expansion under pressure exerted by the fluid within the reservoir. When present, material used in forming a mesh layer 235 may include, without limitation, polyethylene terephthalate, polypropylene, polyamide, polyethylene, and combinations thereof as well as any other material capable of producing an equivalent structure.

### III. Backing Layer

As noted above, the device may also include a backing layer 22, 122, extending across portion of the fluid reservoir 40, 140 opposite the septum 21, 121. The backing layer 22, 122 may be positioned inside the release structure polymer 20 (as shown in FIG. 1 and FIG. 2) or may form an external surface of the device (as shown in FIG. 3 and FIG. 4). The backing layer 22, 122 may be bonded to a portion of the polymer release structure 20, 120 through utilization of an adhesive, ultrasonic welding, etc. or may be attached to the polymer release structure 20, 120 through utilization of an attachment mechanism such as hooks, tabs, etc., or some combination thereof. The backing layer 22, 122 may have a disc shape, and may provide structure to one end of a device, for instance in those embodiments in which the release structure polymer 20, 120 is flexible.

Typically, the backing layer is formed from a “puncture-resistant” material to avoid over-penetration of the syringe needle when refilling the reservoir. The puncture-resistant material may deform when contacted by a needle, but the energy required to pass through the backing layer may be great. A person refilling the reservoir by use of the needle will notice the increased resistance and realize the tip of the needle has contacted the side of the reservoir opposite the septum. Examples of suitable puncture-resistant/materials may include, for instance, polymeric; materials, metals, ceramics, or combinations thereof. Suitable polymeric materials may likewise include polyolefins, polyimides, thermoplastic or thermoset polyurethanes, silicones, acrylonitrile butadiene styrenes, epoxies, rubbers, polyethylene terephthalates, polycarbonates, polyisoprenes, polysulfones, fluoropolymers (e.g., polytetrafluoroethylene), etc. In some embodiments, the backing layer 22, 122 may include a polymeric material that is the form of fibers, such as carbon fibers, glass fibers, quartz fibers, polyester fibers, aramid fibers (e.g., KEVLAR®), etc. The fibers may be in the form of a textile material (e.g., woven fabric, mesh, mat, nonwoven web, etc.) that is formed from such fibers. A metal can be a biocompatible and implantable metal such as, for example, stainless steel, aluminum, titanium, or other metal. The metal can include a metal mesh material having mesh dimensions that are small enough such that a needle (e.g., a 24 gauge needle) cannot pass through it.

In certain embodiments, additional portions (e.g., sidewalls) of the reservoir can also be formed from the same materials as described with regard to the backing layer. For example, the reservoir, excluding the septum, can be formed from with any of the “puncture-resistant” materials as disclosed hereinabove. In a certain embodiment, the reservoir and backing layer can be formed from the same metal material, such as a metal mesh material as described.

In some embodiments, a component of the device, e.g., the backing layer, can include a contrast agent, e.g., barium sulfate, as may be utilized to confirm proper subdermal location of the device following implantation.

### IV. Contraceptive Agent

The contraceptive agent can include any contraceptive agent and can be capable of prohibiting and/or treating a condition, disease, and/or cosmetic state a patient. The contraceptive agent may be prophylactically, therapeutically, and/or cosmetically active, either systemically or locally. The dosage level of the contraceptive agent delivered will vary depending on the particular compound employed and the time period for which it is intended to be released. The dosage level is generally high enough to provide a therapeutically effective amount of the contraceptive agent to render a desired therapeutic outcome, e.g., a level or amount effective to reduce or alleviate symptoms of the condition for which it is administered. The exact amount necessary will vary, depending on the subject being treated, the age and general condition of the subject to which the contraceptive agent is to be delivered, the capacity of the subject's immune system, the degree of effect desired, the severity of the condition being treated, the particular compound selected and mode of administration of the composition, among other factors. An appropriate effective amount can be readily determined by one of skill in the art. For example, an effective amount will typically range from about 5 μg to about 200 mg, in some embodiments from about 5 μg to about 100 mg per day, and in some embodiments, from about 10 μg to about 1 mg of the contraceptive agent delivered per day.

The contraceptive agent can be either naturally occurring or man-made by any method known in the art. Typically, it is also desired that the contraceptive agent is stable at high temperatures so that it can retain desired activity during formation, transport, storage and following insertion into a device reservoir prior to delivery. For example, the contraceptive agent typically remains stable at temperatures of from about 20° C. to about 100° C., in some embodiments from about 30° C. to about 80° C., and in some embodiments, from about 50° C. to about 70° C.

The contraceptive agent may include a progestogen. Suitable progestogens that can be administered using the implantable and refillable device can include progesterone per se, i.e., the active natural progestogen that is found in the corpus luteum, placenta, and adrenal cortex. Other progestogens that can be effectively administered as contraceptive agents using the implantable device include other naturally occurring progestogens, synthetic progestogens, and semi-synthetic progestogens. Synthetic progestogens and semi-synthetic progestogens are generally known in the art as “progestins.” Specific examples of progestogens useful in conjunction with the device can include, without limitation, 21-acetoxypregnenolone, allylestrenol, anagestone (17α-hydroxy-6α-methylpregn-4-en-20-one), anagestone 17α-acetate, chlormadinone, chlormadinone 17α-acetate, chloroethynyl norgestrel, cyproterone, cyproterone 17α-acetate, desogestrel, dienogest, dimethisterone (6α,21-dimethylethisterone), drospirenone (1,2-dihydrospirorenone), ethisterone (17α-ethinyltestosterone or pregneninolone), ethynerone, etynodiol diacetate (norethindrol diacetate), etonogestrel (11-methylene-levo-norgestrel; 3-keto-desogestrel), gestodene, hydroxyprogesterone (17α-hydroxyprogesterone), hydroxyprogesterone caproate, hydroxyprogesterone acetate, hydroxyprogesterone heptanoate, levonorgestrel, lynestrenol, medrogestone (6,17α-dimethyl-6-dehydroprogesterone), medroxyprogesterone, medroxyprogesterone acetate, megestrol, megestrol acetate, segesterone acetate, nomegestrol, nomegestrol acetate, norethindrone (norethisterone; 19-nor-17α-ethynyltestosterone), norelgestromin (17-deacetylnorgestimate), noretynodrel, norgestrienone, progesterone, and retroprogesterone. In some embodiments, the progestogen can include, by way of example only, desogestrel, dienogest, drospirenone, ethisterone, etonogestrel, gestodene, levonorgestrel, medroxyprogesterone, megestrol, norethindrone, norgestimate, or esters of any of the foregoing when the compound allows for esterification (e.g., medroxyprogesterone acetate, megestrol acetate, norethindrone acetate, etc.).

The contraceptive agent can include a combination, e.g., a mixture, of a progestogen and an additional contraceptive agent. An additional contraceptive agent can be an estrogen, i.e., an estrogenic compound. For instance, the ratio of progestogen to estrogen can correspond to the progestogen-to-estrogen ratios of commercially available dual hormone contraceptive products known in the art, regardless of the mode of administration. Suitable estrogenic compounds will be known to those skilled in the art and are described in the literature, and include, without limitation, synthetic and natural estrogens such as: estradiol (i.e., 1,3,5-estratriene-3,17β-diol, or “17β-estradiol”) and its esters (e.g., estradiol benzoate, valerate, cypionate, heptanoate, decanoate, acetate and diacetate), 17α-estradiol, ethinylestradiol (i.e., 17α-ethinylestradiol) and esters and ethers thereof (e.g., ethinylestradiol 3-acetate and ethinylestradiol 3-benzoate, estriol and estriol succinate), polyestrol phosphate, estrone and its esters and derivatives (e.g., estrone acetate, estrone sulfate, and piperazine estrone sulfate), quinestrol, mestranol, and conjugated equine estrogens.

Derivatives and analogs of progestogens and estrogen are encompassed herein. For instance, when the term “cyproterone” is used herein, the agent referred to may be cyproterone per se or a cyproterone ester such as cyproterone 17α-acetate, when the term “medroxyprogesterone” is used, the agent referred to may be medroxyprogesterone per se or a medroxyprogesterone ester such as medroxyprogesterone acetate, etc.

If desired, the contraceptive agent may be provided in the form of a composition, such as a solution, dispersion, suspension, emulsion, etc., as well as a powder that can be reconstituted into a liquid form prior to delivery. Examples of suitable aqueous and nonaqueous carriers, diluents, solvents or vehicles for use in such compositions may include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. Typically, the contraceptive agent will constitute from about 5 wt. % to about 60 wt. %, in some embodiments from about 10 wt. % to about 50 wt. %, and in some embodiments, from about 15 wt. % to about 45 wt. % of the composition, while water and other optional components can constitute from about 40 wt. % to about 95 wt. %, in some embodiments from about 50 wt. % to about 90 wt. %, and in some embodiments, from about 55 wt. % to about 85 wt. % of the composition. The composition may also optionally contain one or more excipients if so desired, such as radiocontrast agents, release modifiers, bulking agents, plasticizers, surfactants, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the composition. Antimicrobial agents and/or preservatives may be employed, for instance, to help prevent surface growth and attachment of bacteria, such as metal compounds (e.g., silver, copper, or zinc), metal salts, quaternary ammonium compounds, etc. The composition can contain minor amounts of auxiliary substances, such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions and by the use of surfactants. The composition also may contain adjuvants such as wetting agents, emulsifying agents and dispersing agents. It also may be desirable to include isotonic agents such as sugars, sodium chloride and the like.

### V. Use of Device

The implantable device may be used in a variety of different ways to treat prohibit and/or treat a condition, disease, or cosmetic state in a patient. The term “implantable device” as used herein, is intended to cover a variety methods of use. For example, the implantable device can be implanted into the body (e.g., subcutaneously) or the implantable device can be inserted into the body (e.g., intravaginally). The device may be implanted using standard techniques. The delivery route may be intrapulmonary, gastroenteral, subcutaneous, intramuscular, intravaginal, intrauterine, or any other suitable delivery route. The device may be placed in a tissue site of a patient in, on, adjacent to, or near an area of the body where delivery is targeted. The device may also be employed together with current systemic active ingredients. The device can also be employed after a patient has been treated with a therapy to ameliorate post-treatment symptoms or side effects. In one particular embodiment, the device can be utilized to deliver a contraceptive agent. For instance, the device may be implanted as an intrauterine or subcutaneous implant to provide highly effective and reversible contraception to a subject in need thereof. In one embodiment, the subcutaneous implant can be inserted subdermally on the inner side of a woman's non-dominant arm as is known with other implantable contraceptive devices. The implantable device can be in different forms, such as an implant (e.g., subcutaneous implant), an intrauterine system (IUS) (e.g., intrauterine device), a helical coil, a spring, a rod, a cylinder, and/or a vaginal ring. For example, single device including the release structure, the septum and the backing layer can be in the form a ring such that the reservoir is likewise in the form of a ring.

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed is:

1. An implantable and refillable device for delivering a contraceptive agent, the device comprising:
a reservoir within which the contraceptive agent is capable of being retained, wherein the reservoir defines a first surface and a second surface opposing the first surface;
a release structure comprising a hydrophobic polymer and surrounding at least a portion of the reservoir, wherein the release structure is in communication with the reservoir such that the contraceptive agent can pass from the reservoir through the release structure;
a septum positioned adjacent to the first surface of the reservoir; and
a backing layer positioned adjacent to the second surface of the reservoir.

2. The implantable and refillable device of claim 1, wherein the device has a generally circular cross-section.

3. The implantable and refillable device of claim 1, wherein the reservoir has a volume of from about 0.5 to about 5 milliliters.

4. The implantable and refillable device of claim 1, wherein the device is in the form of a disc.

5. The implantable and refillable device of claim 1, wherein the hydrophobic polymer comprises a semi-crystalline olefin copolymer.

6. The implantable and refillable device of claim 5, wherein the semi-crystalline copolymer is derived from at least one olefin monomer and at least one polar monomer.

7. The implantable and refillable device of claim 6, wherein the olefin monomer includes ethylene.

8. The implantable and refillable device of claim 6, wherein the polar monomer includes vinyl acetate, vinyl alcohol, maleic anhydride, maleic acid, acrylic acid, methacrylic acid, acrylate, methacrylate, ethyl acrylate, methyl methacrylate, ethyl methacrylate, or a combination thereof.

9. The implantable and refillable device of claim 6, wherein the polar monomer constitutes from about 10 wt. % to about 45 wt. % of the copolymer.

10. The implantable and refillable device of claim 5, wherein the olefin copolymer includes an ethylene vinyl acetate copolymer.

11. The implantable and refillable device of claim 1, wherein the hydrophobic polymer has a melt flow index of from about 0.2 to about 100 grams per 10 minutes as determined in accordance with ASTM D1238-13 at a temperature of 190° C. and a load of 2.16 kilograms.

12. The implantable and refillable device of claim 1, wherein the release structure further includes a hydrophilic compound.

13. The implantable and refillable device of claim 12, wherein the hydrophilic compound includes a hydrophilic polymer.

14. The implantable and refillable device of claim 12, wherein the hydrophilic compound includes a plurality of water-soluble particles distributed within the release structure.

15. The implantable and refillable device of claim 1, wherein the septum comprises a material that exhibits a Shore A hardness of about 40 or less.

16. The implantable and refillable device of claim 1, wherein the septum comprises a nitrile rubber, styrene block copolymer, polyurethane, silicone elastomer, or a combination thereof.

17. The implantable and refillable device of claim 1, wherein the septum comprises multiple layers.

18. The implantable and refillable device of claim 1, wherein the backing layer includes a puncture-resistant material comprising a polymeric material, metal, ceramic, or a combination thereof.

19. The implantable and refillable device of claim 18, wherein the backing layer comprises biocompatible fibers.

20. A contraceptive method, the method comprising implanting the device of independent claim 1 in a patient and filling the reservoir with a contraceptive agent.

21. The method of claim 20, the method of filling the reservoir comprising:
inserting a needle through the septum;
injecting the contraceptive agent into the reservoir through the needle; and
retracting the needle from the septum.

22. The method of claim 20, the contraceptive agent includes a progestogen.

23. The method of claim 22, wherein the contraceptive agent further includes an estrogen.

24. The method of claim 20, further comprising, following a period of time during which the contraceptive agent passes across the release structure, refilling the reservoir.

25. The method of claim 20, wherein the device is implanted as an intrauterine implant or a subcutaneous implant.
